(19) United States
(12) Patent Application Publication
(10) Pub. No.: US 2021/0378924 A1
(43) Pub. Date: Dec. 9, 2021

(54) Oral Care Compositions

(71) Applicant: Colgate-Palmolive Company, New York, NY (US)

(72) Inventors: Manisha JHA, Mumbai (IN); Neelima UTGIKAR, Maharashtra (IN); Shashank POTNIS, Thane (W) (IN); Rolando PLATA, Las Pinas City (PH); Prem SREENIVASAN, Westfield, NJ (US); Fusong SUN, Martinsville, NJ (US)

(73) Assignee: Colgate-Palmolive Company, New York, NY (US)

(21) Appl. No.: 16/642,050

(22) PCT Filed: Apr. 10, 2018
(86) PCT No.: PCT/US2018/026885

§ 371 Date: Feb. 26, 2020

(30) Foreign Application Priority Data
Aug. 31, 2017 (IN) 201741030944

(51) Int. Cl. A61K 8/27; A61K 8/21; A61K 8/36; A61K 8/73; A61K 8/92; A61K 8/9789; A61K 8/98; A61Q 11/00

(52) U.S. Cl. CPC A61K 8/27; A61K 8/21; A61K 8/36; A61K 8/73; A61K 2800/591; A61K 8/9789; A61K 8/988; A61Q 11/00; A61K 2800/92; A61K 8/922

(57) ABSTRACT

Described herein are oral care compositions which contain antibacterial amounts of zinc, a calcium carbonate base; a gum system; and a natural ingredient component.

There is an interest in providing oral care products which have a natural flavor profile. In particular, oral care products containing a mixture of herbal ingredients, such as those reported in Ayurvedic texts.

However, the degree of antibacterial action achieved with herbal ingredients is not as complete as would be desired. It would therefore be desirable to provide such oral care products containing herbal ingredients having enhanced antibacterial effectiveness.

Embodiments of the present invention provide compositions which address, inter alia, this problem.

### BRIEF SUMMARY

In some embodiments, the present invention provides an oral care composition comprising: An oral care composition comprising: a first source of zinc comprising zinc oxide; a second source of zinc comprising zinc citrate; a gum system comprising carrageenan; a calcium source comprising natural calcium carbonate and precipitated calcium carbonate; and herbal extract and essential oil.

Some embodiments provide a method of treating or preventing a disease or condition of the oral cavity comprising contacting an oral cavity surface of a patient in need thereof with any one of the compositions described herein.

### DETAILED DESCRIPTION

It has been surprisingly found that the current formulations offer the advantage of providing a Naturals flavor profile along with a robust antimicrobial protection, without significantly interfering with the stability of the oral care composition and by allowing for formulations which avoid undesirable aesthetic qualities (e.g., poor taste).

The compositions described have a surprisingly advantageous combination of good viscosity and stability of the natural ingredients which was not shared by formulations of similar composition, if they did not include all of the first source of zinc comprising zinc oxide; a second source of zinc comprising zinc citrate; a gum system comprising carrageenan; a calcium source comprising natural calcium carbonate and precipitated calcium carbonate; and herbal extract and essential oil.

In one aspect the invention is an oral care composition (Composition 1.0) comprising:
a, a first source of zinc comprising zinc oxide;
b, a second source of zinc comprising zinc citrate;
c, a gum system comprising carrageenan;
d, a carrier comprising natural calcium carbonate and precipitated calcium carbonate;
e, and at least one herbal extract(s) and at least one essential oil.

For example, the invention contemplates any of the following compositions (unless otherwise indicated, values are given as percentage of the overall weight of the composition).
1.1 Composition 1.0, wherein the ratio of the amount of zinc oxide (e.g., wt. %) to zinc citrate (e.g., wt %) is from 1.5:1 to 4.5:1 (e.g., 2:1, 2.5:1, 3:1, 3.5:1, or 4:1).
1.2 Any of the preceding compositions comprising zinc citrate and zinc oxide, wherein the zinc citrate is in an amount of from 0.25 to 1 wt % (e.g., 0.5 wt. %) and zinc oxide may be present in an amount of from 0.75 to 1.25 wt % (e.g., 1.0 wt. %) based on the weight of the oral care composition.
1.3 Any of the preceding compositions, wherein the zinc citrate is in an amount of from 0.3 to 0.8 wt % (e.g., 0.5 wt. %) and zinc oxide may be present in an amount of from 0.8 to 1.2 wt %⁰ (e.g., 1.0 wt. %) based on the weight of the oral care composition.
1.4 Any of the preceding compositions, wherein the zinc citrate is in an amount of from 0.4 to 0.6 wt %⁰% (e.g., 0.5 wt. %) and zinc oxide may be present in an amount of from 0.9 to 1.1 wt % (e.g., 1.0 wt. %) based on the weight of the oral care composition
1.5 Any of the preceding compositions wherein the zinc citrate is about 0.5 wt % (e.g., zinc citrate trihydrate).
1.6 Any of the preceding compositions wherein the zinc oxide is about 1.0 wt %.
1.7 Any of the preceding compositions where the zinc citrate is about 0.5 wt % and the zinc oxide is about 1.0 wt %.
1.8 Any of the preceding compositions wherein the composition contains one or more herbal extract(s) and one or more essential oils selected from the group consisting of amla extract, honey extract, almond extract, aloe vera extract, maricha extract, ginger extract, fenugreek, neem seed oil, sesame oil, cinnamon leaf oil, clove oil, thyme oil, eucalyptus oil, eugenol, menthol, babool and camphor.
1.9 Any of the preceding compositions wherein the herbal extracts and essential oils are selected from amla extract, honey extract, neem seed oil, basil oil, aloe vera extract, cinnamon leaf oil, clove oil, and camphor.
1.10 Any of the preceding compositions wherein the carrageenan gum system comprises carrageenan concentrate.

As used throughout, ranges are used as a short hand for describing each and every value that is within the range. Any value within the range can be selected as the terminus of the range.

In addition, all references cited herein are hereby incorporated by reference in their entireties.

In the event of a conflict in a definition in the present disclosure and that of a cited reference, the present disclosure controls.

In some embodiments, the compositions further comprise one or more components selected from a fluoride ion source; a tartar control agent; a buffering agent; an antibacterial agent; an abrasive; and a combination of two or more thereof.

Some embodiments provide compositions wherein at least one of the one or more components is a fluoride ion source selected from: stannous fluoride, sodium fluoride, potassium fluoride, sodium monofluorophosphate, sodium fluorosilicate, ammonium fluorosilicate, amine fluoride, ammonium fluoride, and a combination of two or more thereof.

Other optional additives may be included. Among such optional additives, included are those provided in order to change appearance or aesthetic appeal, and/or to preserve the final product, and/or for taste/cosmetic appeal and/or as therapeutic and prophylactic ingredients for oral health, prevention or treatment of a condition or disorder of hard or soft tissue of the oral cavity, or the prevention or treatment of a physiological disorder or condition.

Some embodiments provide a composition wherein a preservative is present. In some embodiments, the preservative is selected from parabens, potassium sorbate, benzyl alcohol, phenoxyethanol, polyaminopropryl biguanide, caprylic acid, sodium benzoate and cetylpyridinium chloride. In some embodiments, the preservative is present at a concentration of about 0.0001 to about 1%, by weight.

Colorants such as dyes may be food color additives presently certified under the Food Drug & Cosmetic Act for use in food and ingested drugs, including dyes such as FD&C Red No. 3 (sodium salt of tetraiodofluorescein), Food Red 17, disodium salt of 6-hydroxy-5-{(2-methoxy-5-methyl-4-sulphophenyl)azo}-2-n-aphthalenesulfonic acid, Food Yellow 13, sodium salt of a mixture of the mono and disulphonic acids of quinophthalone or 2-(2-quinolyl) indanedione, FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphthol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-DELTA-3,5-cycl-ohexadienimine], FD&C Blue No. 1 (disodium salt of dibenzyldiethyl-diamino-triphenylcarbinol trisulfonic acid anhydrite), FD&C Blue No. 2 (sodium salt of disulfonic acid of indigotin) and mixtures thereof in various proportions.

Typically, colorants if included are present in very small quantities.

Flavor profile is provided by various ingredients which include, but are not limited to, herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract) synthetic flavor oils and flavoring aromatics, and/or oils, oleo resins and extracts derived from herbs plants, leaves, flowers, fruits, honey and so forth, and combinations thereof. Representative herbal extracts include: spearmint oil, cinnamon oil, peppermint oil, clove oil, bay oil, thyme oil, cedar leaf oil, oil of nutmeg, oil of sage, and oil of bitter almonds. Examples of herbal extracts include mints such as peppermint, artificial vanilla, cinnamon derivatives, and various fruit flavors, whether employed individually or in admixture. Generally, any flavoring agent or food additive, such as those described in Chemicals Used in Food Processing, publication 1274 by the National Academy of Sciences, pages 63-258, may be used. Typically, the total amount of herbal extract or essential oil is from about 0.1 to about 5%, by weight; from about 0.2 to about 4% by weight; about 0.5 to about 3% by weight; about 1 to about 2.5% by weight; from 1 to about 3% by weight.

A preferred group of herbal extracts and essential oils include amla extract, honey extract, almond extract, aloe vera extract, maricha extract, ginger extract, fenugreek, neem seed oil, sesame oil, cinnamon leaf oil, clove oil, thyme oil, eucalyptus oil, eugenol, menthol, babool and camphor.

Sweeteners include both natural and artificial sweeteners. Suitable sweeteners include water soluble sweetening agents such as monosaccharides, disaccharides and polysaccharides such as xylose, ribose, glucose (dextrose), mannose, galactose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalanine methyl ester (aspartame). In general, the effective amount of sweetener is utilized to provide the level of sweetness desired for a particular composition, will vary with the sweetener selected. This amount will normally be from about 0.001 to about 5%, by weight. In some embodiments, the sweetener is sodium saccharin and is present at a concentration of about 0.01%, by weight.

Whitening agents, material which is effective to effect whitening of a tooth surface to which it is applied, such as hydrogen peroxide and urea peroxide, high cleaning silica, preservatives, silicones, and chlorophyll compounds may be incorporated into the compositions of the present invention. In various embodiments, the compositions of this invention comprise a peroxide whitening agent, comprising a peroxide compound. A peroxide compound is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof. In some embodiments, the peroxide compound comprises hydrogen peroxide. In some embodiments, the peroxide compound consists essentially of hydrogen peroxide. In some embodiments a non-peroxide whitening agent may be provided. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite. One or more whitening agents are optionally present in a tooth-whitening effective total amount. In some embodiments the whitening agent is separated from the aqueous carrier. In some embodiments the whitening agent is separated from the aqueous carrier by encapsulation of the whitening agent.

Optionally, breath freshening agents may be provided. Any orally acceptable breath freshening agent can be used, including without limitation. One or more breath freshening agents are optionally present in a breath freshening effective total amount.

Other embodiments provide compositions wherein at least one of the one or more components is a tartar control agent. Tartar control agents among those useful herein include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J. In some embodiments, a phosphate is present at a concentration of from about 0.01 to about 10%, by weight. In some embodiments, a phosphate is present at a concentration of from about 1%, by weight.

Some embodiments provide compositions wherein a buffering agent is present. In some embodiments, sodium phosphate monobasic is present at a concentration of from about 0.01 to about 5%, by weight. In some embodiments, sodium phosphate monobasic is present at a concentration of about 1%, by weight. In some embodiments, sodium phosphate dibasic is present at a concentration of from about 0.01 to about 5%, by weight. In some embodiments, sodium phosphate dibasic is present at a concentration of about 0.15%, by weight.

Antioxidants are another class of optional additives. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Also optional, a saliva stimulating agent, useful for example in amelioration of dry mouth, may be included. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective total amount.

Optionally, an antiplaque (e.g., plaque disrupting) agent may be included. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and mixtures thereof.

Optional desensitizing agents include potassium citrate, potassium chloride, potassium tartrate, potassium bicarbonate, potassium oxalate, potassium nitrate, strontium salts, and mixtures thereof.

Optional additives also include vitamins, and proteins. Vitamins include Vitamins C and D, thiamine, riboflavin, calcium pantothenate, niacin, folic acid, nicotinamide, pyridoxine, cyanocobalamin, para-aminobenzoic acid, bioflavonoids, pantheon, retinyl palmitate, tocopherol acetate, and mixtures thereof. Suitable proteins include milk proteins and enzymes such as peroxide-producing enzymes, amylase, plaque-disrupting agents such as papain, glucoamylase, glucose oxidase, and “next generation” enzymes.”

In some embodiments, the composition has a free water content of greater than about 10%, by weight. In some embodiments, the composition has a free water content of greater than about 11%, by weight. In other embodiments, the composition has a free water content of greater than about 12%, by weight. Yet other embodiments provide compositions wherein the free water content is greater than about 13%, by weight. Still other embodiments provide compositions having a free water content of greater than about 14%, by weight. In some embodiments, the composition has a free water content of greater than about 15%, by weight. While other embodiments provide compositions have a free water content of greater than about 16%, by weight. In some embodiments, the composition has a free water content of about 17%, by weight. In some embodiments, the composition has a free water content of greater than about 17%, by weight. In some embodiments, the composition has a free water content of from about 10% to about 20%, by weight.

Some embodiments provide a method of treating or preventing a disease or condition of the oral cavity comprising contacting an oral cavity surface of a patient in need thereof with any one of the compositions described herein. In other embodiments, the disease or condition of the oral cavity is halitosis. In some embodiments, the present invention provides a method of reducing volatile sulfur compounds in the oral cavity of a subject in need thereof. In further embodiments, the present invention provides a method for increasing the delivery of a metal ion to an oral cavity surface.

In certain embodiments, the compositions described herein can be used, for example, for cavity prevention, whitening, plaque prevention or reduction, gingivitis prevention or reduction, tartar control, breath malodor prevention or reduction, and stain prevention.

The specific composition of the carrier preferably depends on the intended use of the composition. In various embodiments, the carrier is aqueous, comprising from about 5 to about 95%, by weight, water or from about 10 to about 70%, by weight, water. In other embodiments, the carrier is substantially non-aqueous. In a dentifrice carrier, water content can be from about 5 to about 70%, from about 10 to about 50%, or from about 20 to about 40%, by weight.

The carrier may comprise any of a variety of materials, including emulsifiers, thickeners, fillers, and preservatives. In some embodiments, the carrier may include a functional or active material, such as those described above.

In some embodiments, the carrier comprises a humectant, such as glycerin, sorbitol or an alkylene glycol such as polyethylene glycol or propylene glycol. In some embodiments, the carrier comprises a humectant at a level of from about 10 to about 80% by weight, or about 20 to about 60% by weight of the composition. Carrier compositions among those useful herein are disclosed in U.S. Pat. No. 5,695,746 to Garlick, Jr., et al, and U.S. Pat. No. 4,839,157 to Mei-King Ng et al.

Thickeners or gelling agents useful herein include inorganic, natural or synthetic thickeners or gelling agents. In some configurations, the carrier comprises the thickener and gelling agent at total levels of from about 0.1 to about 15% by weight, or from about 0.4 to about 10% by weight of the composition. Examples of thickeners and gelling agents useful herein include inorganic thickening silicas such as: an amorphous silica, for example Zeodent® 165 (Huber Corporation); Irish moss; iota-carrageenan; gum tragacanth; or polyvinylpyrrolidone. The preferred thickener is carrageenan gum, particularly carrageenan concentrate. In some embodiments, the amount of carrageenan concentrate is from about 0.5% to about 2% by weight; from about 0.5 to about 1.5% by weight, about 0.7 to about 1.2% by weight.

In certain embodiments, the carrier comprises an abrasive or polishing agent comprising a combination of refined natural calcium carbonate and precipitated calcium carbonate. Optional abrasive include a calcined alumina, sodium bicarbonate, dicalcium phosphate or calcium pyrophosphate. In various embodiments, the carrier is clear. In various embodiments, the carrier comprises an abrasive at a level of from about 5 to about 70% by weight of the composition; from about 20 to about 60% by weight; from about 30 to about 50 percent by weight; from about 35 to about 45% by weight.

In some embodiments, the compositions comprise a surfactant or mixture of surfactants. Surfactants among those useful herein include water-soluble salts of at least one higher fatty acid monoglyceride monosulfate, such as the sodium salt of the monosulfated monoglyceride of hydrogenated coconut oil fatty acids; cocamidopropyl betaine; a higher alkyl sulfate such as sodium lauryl sulfate, an alkyl aryl sulfonate such as sodium dodecyl benzene sulfonate; a higher alkyl sulfoacetate; sodium lauryl sulfoacetate; a higher fatty acid ester of 1,2-dihydroxy propane sulfonate; and a substantially saturated higher aliphatic acyl amides of a lower aliphatic amino carboxylic acid, such as those having 12 to 16 carbons in the fatty acid, alkyl or acyl radicals; and mixtures thereof. Amides can be, for example, N-lauroyl sarcosine, and the sodium, potassium, and ethanolamine salts of N-lauroyl, N-myristoyl, or N-palmitoyl sarcosine. In various embodiments, the surfactant is present at a concentration of from about 0.3 to about 5% by weight of composition, or about 0.5 to about 3% by weight of composition.

Compositions as described herein can be prepared according to methods readily known to those skilled in the art.

Embodiments of the present invention are further described in the following examples. The examples are merely illustrative and do not in any way limit the scope of the invention as described and claimed.

### EXAMPLES

A series of formulations is prepared and evaluated on a range of physical characteristics, and display unacceptable performance either as initially formulated or upon storage under the stated conditions, as detailed below.

### Comparative Example A

A composition with a chalk (calcium carbonate) backbone is prepared for use in Comparative Example A as described in Table 1 (below).

TABLE 1

| Ingredient | Wt. % |
|---|---|
| Humectant (70%) Solution | 18.00 |
| Abrasive | 1.00 |
| Purified water | 26.38 |
| CarbobxyMethyl Cellulose TMS | 1.00 |
| Sweetener | 0.22 |
| Refined Natural Calcium Carbonate | 35.00 |
| Precipitated Calcium Carbonate | 9.00 |
| Abrasive | 0.75 |
| Sodium Monofluorophosphate-USP | 0.76 |
| pH Adjusting Agent | 0.50 |
| Anionic Surfactant | 2.00 |
| Zinc Oxide | 1.00 |
| Zinc Citrate | 0.50 |
| Potassium Nitrate | 0.50 |
| Benzyl Alcohol | 0.30 |
| Flavor (containing essential oils) | 1.80 |
| Amla Extract | 1.00 |
| Honey Extract | 0.0004 |
| Almond Extract | 0.0004 |
| Neem Seed Oil | 0.010 |
| Aloe Vera Extract | 0.0004 |
| Sesame Oil | 0.0004 |
| Cinnamon leaf oil | 0.0004 |
| Colorants | 0.28 |
| TOTAL | 100.0 |

Physical properties of the compositions are tested upon completion of formulating at 25° C., and after storage for the time period indicated, under the stated conditions of temperature and relative humidity (R.H.). Upon initial testing, the Brookfield viscosity of these slurry composition was measured and the values reported are expressed as bku, which is a machine based measurement of relative resistance to deformation, used here as a shorthand comparator within these slurry systems. On the bku scale, the desired viscosity for toothpaste in the Naturals segment is around 25-30 bku. The viscosity of this sample is observed to be undesirably high (above 50 bku). The product with this formula is therefore difficult to squeeze from a tube. In addition, discoloration in the formula is observed, and separation of herbal extracts is observed. Upon storage, the product is consistently hard to squeeze due to high viscosity, and separation of the composition was observed, as described in Table 2 (below).

TABLE 2

| | | | Viscosity | Physical Examination |
|---|---|---|---|---|
| Conditions | Time | | (bku) | of Dental Cream |
| 25° C. | 0 | mo | 50.4 | PASS, Acceptable |
| 25° C./60% RH | 1 | mo | 48 | PASS, Acceptable |
| 25° C./60% RH | 2 | mo | 52.7 | Not Acceptable Appearance, |
| | | | | Separation |
| 25° C./60% RH | 3 | mo | 50.0 | Not Acceptable, Appearance, |
| | | | | Hard to squeeze, Amla |
| | | | | separation, Discoloration |
| | | | | due to Amla separation |
| 40° C./75% RH | 1 | mo | 47 | PASS, Acceptable |
| 40° C./75% RH | 2 | mo | 49.8 | Not Acceptable Appearance, |
| | | | | Separation |
| 40° C./75% RH | 3 | mo | 48.3 | Not Acceptable Appearance, |
| | | | | Hard to squeeze, Amla |
| | | | | separation, Discoloration |
| | | | | due to Amla separation |
| 49° C. | 4 | wk | — | PASS, Acceptable |
| 4° C. | 24 | hr | — | PASS, Acceptable |

### Comparative Example B

A composition with silica backbone is prepared for use in Comparative Example B and formulated as described in Table 3 (below).

TABLE 3

| Ingredient | Wt. % |
|---|---|
| Humectant (70%) | 68.00 |
| PEG 600 | 1.00 |
| Carboxymethyl Cellulose | 0.50 |
| Sweetener | 0.15 |
| Purified Water | 6.15 |
| Sodium Fluoride | 0.22 |
| Maricha (Piper nigrum) | 0.70 |
| Shunthi/Dry Ginger extract (Zingiber | 0.70 |
| officinale) | |
| Clove Oil | 0.30 |
| Thymol | 0.20 |
| Eucalyptus Oil | 0.030 |
| Eugenol | 0.20 |
| Babool | 0.50 |
| Fenugreek | 0.10 |
| Karpura/Camphor (Cinnamomum Camphora) | 0.40 |
| Zinc Oxide | 1.00 |
| Zinc Citrate | 0.50 |
| Amla Extract | 0.20 |
| Colorants | 0.35 |
| Abrasive | 8.0 |
| Thickener | 8.0 |
| Anionic Surfactant | 2.0 |
| Menthol | 0.50 |
| Benzyl Alcohol | 0.30 |
| TOTAL | 100.0 |

The composition is evaluated and the results are reported in Table 4 (below).

TABLE 4

| | | | Viscosity | Physical Examination |
|---|---|---|---|---|
| Condition | Time | | (bku) | of Dental Cream |
| 25° C. | 0 | mo | 9 | Acceptable for physical |
| | | | | parameters |
| 25° C./60% RH | 1 | mo | 12 | Low on stand-up, Acceptable |
| | | | | for physical parameters |
| 25° C./60% RH | 2 | mo | 21.24 | Acceptable |
| 25° C./60% RH | 3 | mo | 30.39 | Hard to Squeeze, Acceptable |
| 25° C./60% RH | 6 | mo | 48.53 | Acceptable |
| 40° C./75% RH | 1 | mo | 82 | Acceptable for physical |
| | | | | parameters |
| 40° C./75% RH | 2 | mo | EEE | Difficult to squeeze, Separation |
| | | | | FAIL |
| 40° C./75% RH | 3 | mo | Above | Hard to Squeeze, Separation |
| | | | 100 | |
| 49° C. | 4 | wk | — | Acceptable for physical |
| | | | | parameters |
| 4° C. | 24 | hr | — | Acceptable for physical |
| | | | | parameters |

The results presented in Table 4 (above) show that this formula has very low initial viscosity (around 9 bku) which leads to the composition failing to stand up, and instead flowing off a target surface such as a toothbrush. Upon storage at higher temperatures, the viscosity of the formulation is observed to increase beyond 80 bku.

Based upon the results observed, it is concluded that the formulations containing herbal extracts are not stable in compositions having a silica backbone. Also, it is observed that carboxymethyl cellulose gum system did not produce desired viscosity in both the chalk and silica backbone.

### Comparative Example C

Based upon the observations above, a formulation with Carrageenan Gum system and a water-soluble form of Zinc (Zinc Sulphate) is prepared as shown in Table 5 (below).

TABLE 5

| Ingredient | Wt. % |
|---|---|
| Humectant (70%) | 17.00 |
| Carrageenan Concentrate | 0.950 |
| Sweetener | 0.150 |
| Purified Water | 30.0 |
| pH Adjusting Agent | 0.50 |
| Maricha (Piper nigrum) | 0.70 |
| Shunthi/Dry Ginger extract (Zingiber officinale) | 0.70 |
| Clove Oil | 0.30 |
| Babool | 0.50 |
| Karpura/Camphor (Cinnamomum Camphora) | 0.50 |
| Thymol | 0.20 |
| Eucalyptus Oil | 0.03 |
| Eugenol | 0.17 |
| Fenugreek | 0.10 |
| Zinc Sulphate | 2.20 |
| Amla Extract | 0.20 |
| Ca Carbonate (RNCC) | 43.0 |
| Anionic Surfactant | 2.0 |
| Menthol | 0.50 |
| Benzyl Alcohol | 0.30 |
| TOTAL | 100.0 |

The composition is evaluated and the results are reported in Table 6 (below).

TABLE 6

| | | | Viscosity | Physical Examination |
|---|---|---|---|---|
| Condition | Time | | (bku) | of Dental Cream |
| 25 C. | 0 | mo | 16.9 | Stand up, Less viscous appearance, |
| | | | | Acceptable |
| 25° C./60%RH | 1 | mo | 18.4 | Acceptable for physical parameters |
| 25° C./60%RH | 2 | mo | 14.7 | Acceptable |
| 25° C./60%RH | 3 | mo | 16.08 | Acceptable |
| 40° C./75%RH | 1 | mo | 18.9 | Acceptable for physical parameters |
| 40° C./75%RH | 2 | mo | 13.1 | Acceptable |
| 40° C./75%RH | 3 | mo | 13.50 | Separation, Acceptable |
| 49° C. | 4 | wk | — | Discoloration, Acceptable for |
| | | | | physical parameters |
| 4° C. | 24 | hr | — | Stand up, Less viscous |
| | | | | Appearance, Acceptable |

The results presented in Table 6 (above) show that this formula has a low initial viscosity (around 17 bku) which can lead to the composition failing to stand up, developed some discoloration and separation upon storage stability testing.

### Comparative Example D

A composition with Carrageenan Gum, Zinc Sulphate. Zinc Oxide and herbal extracts is prepared, as described in Table 7 (below).

TABLE 7

| Ingredient | Wt. % |
|---|---|
| Humectant (70%) | 17.0 |
| Carrageenan Concentrate | 0.95 |
| Sweetener | 0.15 |
| Purified Water | 28.95 |
| pH Adjusting Agent | 0.50 |
| Maricha (Piper nigrum) | 0.70 |
| Shunthi (Zingiber officinale) | 0.70 |
| Clove Oil | 0.30 |
| Babool | 0.50 |
| Karpura (Cinnamomum Camphora) | 0.50 |
| Thymol | 0.10 |
| Eucalyptus Oil | 0.03 |
| Eugenol | 0.17 |
| Fenugreek | 0.10 |
| Zinc Sulphate | 1.10 |
| Zinc Oxide | 0.50 |
| Amla Extract | 0.20 |
| Thickener | 1.75 |
| Ca Carbonate (RNCC) | 43.0 |
| Anionic Surfactant | 2.0 |
| Menthol | 0.50 |
| Benzyl Alcohol | 0.30 |
| TOTAL | 100.0 |

The composition is evaluated and the results are reported in Table 8 (below).

TABLE 8

| | | | Viscosity | Physical Examination |
|---|---|---|---|---|
| Condition | Time | | (bku) | of Dental Cream |
| 25° C. | 0 | mo | 16.9 | Stand up, Less viscous |
| | | | | appearance, Acceptable |
| 25° C./60% RH | 1 | mo | 21.5 | Acceptable for physical |
| | | | | parameters |
| 25° C./60% RH | 2 | mo | 19.4 | Acceptable |
| 25° C./60% RH | 3 | mo | 21.67 | Acceptable |
| 25° C./60% RH | 6 | mo | 24.80 | Pass |
| 40° C./75% RH | 1 | mo | 19.5 | Acceptable for physical |
| | | | | parameters |
| 40° C./75% RH | 2 | mo | 26.3 | Acceptable |
| 40° C./75% RH | 3 | mo | 37.74 | Shoulder separation, Acceptable |
| 49° C. | 4 | wk | — | Discoloration, Acceptable for |
| | | | | physical parameters |
| 4° C. | 24 | hr | — | Acceptable |

The results presented in Table 8 (above) show that this formula has a low initial viscosity (around 17 bku) which can lead to the composition failing to stand up, developed some discoloration and separation upon storage stability testing. After storage at elevated temperature, the formula displayed an increase in viscosity, shoulder separation and discoloration.

### Example 1

A composition containing Carrageenan Gum, Zinc Citrate Trihydrate, Zinc Oxide, Calcium Carbonate base, Herbal Extracts and Essential Oils was prepared, as described in Table 9 (below).

TABLE 9

| Ingredient | Wt. % |
|---|---|
| Humectant (70%) | 30.00 |
| Carrageenan Concentrate | 0.825 |
| Sweetener | 0.22 |
| Sodium Monofluorophosphate-USP | 0.76 |
| pH Adjusting Agent | 0.50 |
| Purified Water | 18.15 |
| Thickener | 1.75 |
| Refined Natural Calcium Carbonate | 19.00 |
| Precipitated Calcium Carbonate | 21.50 |
| Annionic Surfactant | 2.00 |
| Zinc Oxide | 1.00 |
| Zinc Citrate Trihydrate | 0.50 |
| Desensitizing agent | 0.30 |
| Abrasive | 1.00 |
| Amla Extract | 0.10 |
| Honey Extract | 0.0004 |
| Neem Seed Oil | 0.010 |
| Fennel Oil | 0.0004 |
| Basil Oil | 0.0004 |
| Aloe Vera Extract | 0.0004 |
| Cinnamon Leaf Oil | 0.0004 |
| Flavor Option 1- Herbal 5(containing | 1.80 |
| essential oils) | |
| Colorants | 0.28 |
| Benzyl Alcohol | 0.30 |
| Total | 100.0 |

The composition described on Table 9 was evaluated and the results are reported in Table 10 (below).

TABLE 10

| | | Viscosity | Physical Examination |
|---|---|---|---|
| Condition | Time | (bku) | of Dental Cream |
| 25° C. | 0 mo | 28 | Acceptable |
| 25° C./60% RH | 1 mo | 26.11 | Acceptable |
| 25° C./60% RH | 2 mo | 28 | Acceptable |
| 25° C./60% RH | 3 mo | 25.3 | Acceptable |
| 40° C./75% RH | 1 mo | 23.68 | Acceptable |
| 40° C./75% RH | 2 mo | 24 | Acceptable |
| 40° C./75% RH | 3 mo | 25.9 | Acceptable |

The results described in Table 10 (above) demonstrate that the composition described in Table 9 formula was stable with respect to all the analytical parameters. This chalk base formulation, containing Calcium Carbonate, Carrageenan, Zinc Citrate Trihydrate, Zinc Oxide, and Herbal Extracts and Essential Oil produced a surprisingly stable, efficacious formula having a Natural connotation of taste and color.

### Efficacy Testing

An in vitro bridging study on planktonic bacteria is conducted to compare the antibacterial efficacy of two different flavor compositions, based upon the composition of Example 1, against a Naturals placebo toothpaste without Zinc, and a toothpaste containing Zinc Oxide and Zinc Citrate without Herbs or Essential Oils (Comparative Example E).

The active ingredients in the Naturals Honey & Naturals Cinnamon are shown in Table 11 (below).

TABLE 11

| Active Antibacterial Ingredient | Wt. % |
|---|---|
| Zinc Oxide | 1 |
| Zinc Citrate | 0.5 |
| Amla Extract | 0.1 |
| Honey Extract | 0.0004 |
| Neem Seed Oil | 0.01 |
| Fennel Oil | 0.0004 |
| Basil Oil | 0.0004 |
| Aloe Vera Extract | 0.0004 |
| Cinnamon Leaf Oil | 0.0004 |
| Flavor (containing essential oils) | 1.8 |

Different essential oils components are used in Naturals Honey & Naturals Cinnamon, in order to produce a different Key signature note:
Example 1—Naturals Honey: Clove, Camphor, Honey
Example 2—Naturals Cinnamon: Clove, Camphor, Cinnamon

The Naturals Placebo does not contain Zinc Citrate, Zinc Oxide, Herbal Extracts or Essential oils. The results are reported in Table 12 (below).

TABLE 12
Fluorescence values used as measure of % Viability

| Samples | N | Mean | Grouping |
|---|---|---|---|
| Naturals Placebo | 3 | 74.58 | A |
| Ex. 1 - Naturals Honey | 3 | 18.17 | B |
| Ex. 2 - Naturals Cinnamon | 3 | 19.76 | B |
| Comparative Example E | 3 | 18.14 | B |

The data presented in Table 12 (above) show that the compositions of Example 1 and Example 2 significantly outperform a Naturals Placebo in controlling planktonic bacteria (Note that the Group values indicate statistically significant difference between results, i.e. a Group A value shows a high degree of bacterial viability, a Group B value shows a significantly lower level of bacterial viability).

### Example 3: Consumer Perception on the Product

Consumers evaluated compositions prepared using the Naturals Honey flavored composition described above, as for Example 3, which has key flavor notes of Clove, Camphor & Honey and it was found to be authentic and natural by the consumers, also no metallic or Zinc taste was reported by consumers. This flavor was able to mask the high percentage of Zinc in the formula and at the same time produced a consumer perception of Natural flavor notes.

As those skilled in the art will appreciate, numerous changes and modifications may be made to the embodiments described herein without departing from the spirit of the invention. It is intended that all such variations fall within the scope of the appended claims.

I/We claim:

1. An oral care composition comprising:
a first source of zinc ions;
a second source of zinc ions;
a gum system;
an orally acceptable carrier comprising calcium carbonate; and
a natural ingredient component comprising:
an herbal extract; and
an essential oil.

2. The oral care composition according to claim 1, wherein the first source of zinc ions comprises zinc oxide.

3. The oral care composition according to claim 2, wherein the second source of zinc ions comprises zinc citrate.

4. The oral care composition according to claim 3, comprising:
from about 0.1 to about 5%, by weight, of zinc oxide; and
from about 0.05 to about 2%, by weight, of zinc citrate.

5. The oral care composition according to claim 3, comprising:
from about 0.3 to about 4%, by weight, of zinc oxide; and
from about 0.1 to about 1.5%, by weight, of zinc citrate.

6. The oral care composition according to claim 3, comprising:
from about 0.5 to about 2%, by weight, of zinc oxide; and
from about 0.2 to about 0.75%, by weight, of zinc citrate.

7. The oral care composition according to claim 3, comprising:
about 1%, by weight of zinc oxide; and
about 0.5%, by weight of zinc citrate.

8. The oral care composition according to claim 1, wherein the herbal extract is selected from amla extract, honey extract, almond extract, aloe vera extract, maricha extract, ginger extract, fenugreek, and combinations of two or more thereof.

9. The oral care composition according to claim 1, wherein the essential oil is selected from neem seed oil, sesame oil, cinnamon leaf oil, clove oil, thyme oil, eucalyptus oil, eugenol, menthol, babool, camphor, and combinations of two or more thereof.

10. The oral care composition according to claim 3, wherein the herbal extract is selected from amla extract, aloe vera extract, honey extract and a combination thereof, and the essential oil is selected from neem seed oil, basil oil, cinnamon leaf oil, clove oil, camphor and a combination of two or more thereof.

11. The oral care composition according to claim 1, wherein the gum system comprises carrageenan.

12. The oral care composition according to claim 10, wherein the gum system comprises carrageenan concentrate.

13. The oral care composition according to claim 1, further comprising a fluoride ion source selected from: stannous fluoride, sodium fluoride, potassium fluoride, sodium monofluorophosphate, sodium fluorosilicate, ammonium fluorosilicate, amine fluoride, ammonium fluoride, and a combination of two or more thereof.

14. The oral care composition according to claim 1, wherein the calcium carbonate is selected from natural calcium carbonate, precipitated calcium carbonate, and combinations thereof.

15. A method of treating or preventing a disease or condition of the oral cavity comprising contacting an oral cavity surface of a patient in need thereof with the oral care composition according to claim 10.

16. The method according to claim 15, wherein the disease or condition of the oral cavity is a disease or condition caused by oral bacteria.
